(19) United States
(12) Patent Application Publication
(10) Pub. No.: US 2015/0342861 A1
(43) Pub. Date: Dec. 3, 2015

(54) HAIR PRODUCT WITH STABLE VISCOSITY

(71) Applicant: The Dial Corporation, Scottsdale, AZ (US)

(72) Inventor: Jennifer Harris, Phoenix, AZ (US)

(73) Assignee: The Dial Corporation

(21) Appl. No.: 14/810,545

(22) Filed: Jul. 28, 2015

Related U.S. Application Data
(63) Continuation-in-part of application No. 14/054,805, filed on Oct. 15, 2013, now abandoned.

(51) Int. Cl. A61K 8/81; A61K 8/34; A61K 8/73; A61Q 5/06

(52) U.S. Cl. CPC A61K 8/8176; A61Q 5/06; A61K 8/345; A61K 8/731; A61K 8/8182; A61K 2800/592; A61K 2800/594

(57) ABSTRACT

A composition of matter, a hair product formulation, comprising three polymer species, one or more polyols, an emulsifier, and water. The formulation balanced between viscosity increasing components and viscosity decreasing components such that the viscosity is stabilized. The emulsifier being used to tune the viscosity of the solution.

### CROSS-REFERENCES TO RELATED APPLICATIONS

This is a continuation-in-part of U.S. application Ser. No. 14/054,805, filed Oct. 15, 2013, the entire contents and substance of which are hereby incorporated in total by reference.

### FIELD OF THE INVENTION

The present invention generally relates to polymer containing solutions, and more particularly relates to hair products.

### BACKGROUND OF THE INVENTION

Many personal care products require a wide variety of difficult to quantify properties for consumer acceptance. For hair styling products, these properties can include elements like hold, stiffness, drying time, hand feel, viscosity, ease of rinsing (from hands), and ease of rinsing (from hair) after drying. The more properties that need to be optimized for a formulation impact the degrees of freedom in designing a composition.

Accordingly, it is desirable to develop formulations for hair products that control the viscosity, both as formulated and over a shelf life and use life of the product. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A formulation for a hair product is disclosed, the formulation includes three polymer species, one or more polyols, an emulsifier, and water. The proportions of the components are selected such that the components that increase the viscosity are balanced with the components that decrease the viscosity.

A method is provided for enhancing the viscosity stability of a formulation. The method consists of determining the contributions to viscosity over time of the components of the solution and adjusting the concentrations of the components to balances the contributions from components that increase the viscosity of the formulation with the contributions from components that decrease the viscosity of the formulation.

### BRIEF DESCRIPTION OF THE DRAWING

The present invention will hereinafter be described in conjunction with the following drawing figure.

FIG. 1 shows graphs of viscosity behavior over time for some of the compositions described in Table 1 according to some embodiments of this disclosure. The horizontal axes are time in weeks. The vertical axes is viscosity in centistokes (cSt).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Many producers of products seek to minimize the number of ingredients used in formulations. Reducing the number of components may reduce the costs for the producer. For instance, it may reduce the number of suppliers that need to be qualified. It may reduce the quantity and value of supplies that need to be kept on hand as well as the associated storage costs. Reducing the number of components may reduce regulatory or commercial risks. However, reducing the number of components is not without difficulties. For instance, reducing the number of components reduces the number of variables that can be adjusted in a formulation and limits the properties that can result. At some point, the remaining components will not be able to achieve all the properties desired of a formulation. At that point, a determination needs to be made whether the benefits from reduced numbers of components is worth more than the reduced properties.

One area of concern for hair products is viscosity. Viscosity impacts subjective measurements of the product, such as hand feel. It also can impact design of the delivery container to make a product easier or harder to dispense. Viscosity may impact the hold of hair gels.

Viscosity is a difficult property to model from composition and first principles. Modeling can be more challenging in high viscosity formulations where small changes in availability of species, especially of small molecular weight components, can have relatively large impacts on the viscosity. Viscosity can be impacted by the relative amounts of the constituents, the quantity of solvent(s), the tendency to form domains in the mixture, the tendency to bond or organize small molecules (especially water and alcohols that act as solvents); the stability of the mixture and tendency to separate. Accordingly, while examples and explanations are provided in the specification, they represent one understanding of the claimed system and are not intended to limit the scope of the claimed invention.

Viscosity can be measured using a variety of different methodologies. One widely used measurement method involves measuring the sheer force on a turning cylinder in a container, the container being filled with the fluid being measured. Examples of this method include ASTM D2556 Test Method for Apparent Viscosity of Adhesives Having Shear-Rate-Dependent Flow Properties. See also, ISO2555 Plastics—Resins in the Liquid State or as Emulsions or Dispersions—Determination of Apparent Viscosity by the Brookfield Test Method. Other methods include flow rates such as used in ASTM D 445, D1084, D1200, and D1545 (bubble test). Additionally, industry specific methods and proprietary methodologies are sometimes used to quantify viscosity.

Viscosity may depend on sheer rate. Some solutions are sheer thinning meaning that as the sheer rate increases they become less viscous. Other solutions are sheer thickening such that the faster the sheer rate, the greater the viscosity. Finally, some fluids are Newtonian, meaning their viscosity is independent of sheer rate. Many real life fluids and compositions deviate significantly from the Newtonian ideal. Thus, selecting a sheer rate that reflects use conditions is helpful when discussing the viscosity properties of a composition.

Viscosity of a composition can change over time. In some instances, the composition may change due to evaporation. Small molecules tend to evaporate more easily than larger molecules. As a result, small molecules like water and short chain alcohols which function as solvents may preferentially evaporate, leaving larger molecules behind. The loss of smaller molecules will generally result in increases in viscosity.

Another way compositions can change viscosity over time is by undergoing chemical reactions. For instance, a freshly mixed two part epoxy may be fluid but as the components react with each other, the molecular weight increases, becoming more and more viscous. Eventually, the adhesive becomes a solid due to the combination of evaporation and chemical reaction. Chemical reactions can also reduce the viscosity of a solution over time. For instance, mixing carbohydrates and the appropriate enzyme will cause the long carbohydrate molecules to break down into smaller sugars. With polymer solutions, the impact of reactions is not always predictable in their impact on viscosity. For instance, ionizing radiation may cause the breakdown of polymer molecules as well as cross-linking between polymer molecules. Which effect predominates depends on the rates of the various reactions. Thus similar conditions can increase or decrease the viscosity of polymer containing solutions.

Similar to chemical reactions is association of molecules in solution due to intramolecular attraction due to polar, hydrogen bonding, van der Waals, and similar interactions. During mixing, the small molecules such as water may become associated with charged portions of other components. However, because mixing adds energy to the solution, this distribution may not be a thermodynamically stable state. Instead, over time water and other small molecules associated with some components may migrate to associate with other components of the solution. Similarly, state transitions such as precipitation or crystallization can change the viscosity. For instance, precipitates may no longer contribute to the solution properties (or at a reduced level compared to solvated molecules). Crystallization of polymers can produce bonds between polymer chains that act as reversible crosslinks, increasing the viscosity of the solution (for instance in gelatin or agar). Crystallization of non-polymer species tends to function like precipitation and since that tends to indicate the solution is saturated with respect to the precipitating species.

Water is known to organize around charges in solution, with stronger charges forming larger spheres of loosely organized water. The organized water has two competing effects on the mobility of the associate molecule, first it increases the effective size of the associate molecule reducing the migration rate of the molecule and second it tends to make the molecule less prone to interaction with other molecules due to the layer of water surrounding it. Accordingly, the layer of water can increase or decrease the mobility of the associate molecule in solution.

The described impact is greater on small ions or molecules than large ones. For instance the water associated with a long polymer has effectively no increase in the diffusional cross-section of the molecule, in part because the polymer is massive compared to small molecules so a small layer of associated water doesn't significantly impact its mobility. In contrast, a sodium ion may a have its diffusion cross section increase several times, because it is a small molecule with a relative high charge density.

A composition with long polymer chains that have associated water molecules may give up those water molecules to stronger charged smaller molecules. This, in turn, may impact the viscosity of the solution by changing the diffusion rate of the smaller molecules. If the viscosity of the solution is dominated by the lubricity of the small molecule species, then such an arrangement may provide a more stable viscosity compared with other compositions.

In one example, this understanding can be used to form a reservoir of loosely bound water on the polymer chains in a formulation. The bound water has reduced impact the viscosity of the solution because of the size of the polymer species. The formulation also includes smaller, charged or polar molecules that provide the bulk of the mobility in the formulation. In some examples, these smaller molecules are polyols. In other examples, these smaller molecules include choloro-, fluoro-, or thio- containing species as well as amides and amines. At least some of the smaller molecules have a higher affinity for water than the polymers. For instance, the small molecules may be more polar than the polymer. This allows transfer of water from the polymer to the smaller molecules to control the availability of water in the solution. In some examples, the solution contains less water than in necessary to saturate all the components and more water than is necessary to saturate the small molecules. Under these conditions, the small molecules diffusion properties remain relatively constant and the remainder of the water is associated with the polymers.

This approach allows a relatively stable amount of free water to be maintained in a solution such that even high viscosity solutions can be stabilized against drift due to variation in total water in the solution. One way of conceptualizing this is that the water loosely associated with the polymer acts as a buffer to regulate the available free water and water associated with the small molecules. If excess free water is available, it is sequestered with the polymer where it has a minimal impact on viscosity. If the free water leaves the solution (for instance by evaporation), some of the water is replaced from the polymers as the small molecules have greater affinity for water, so that the viscosity impact of changes in water concentration is minimized.

Accordingly, there needs to be a balancing of the hydrophilic polymers and the small molecules. However, the particular small molecules and their concentration can be selected so as to adjust the viscosity of the solution. As described above, the use of polyols is preferred but not required. Polyols provide some advantages that may not be found in all useful species. Polyols are a good choice for this invention because they exist in a range of sizes from very small to longer chains, they have widespread commercial availability, they are high biocompatibility and low regulatory risk (often with well-established histories and are GRAS, Generally Recognized As Safe by the FDA). Polyols solubilize readily in water when it is time to remove a product from skin or hair. Polyols have high water solubility. Some of these features may be less available, depending on the particular species, for the larger category of small molecules with suitable hydrophilic behavior. Accordingly, while polyols are preferred, the disclosed, enabled compositions are not limited by, for instance, regulatory history with the FDA of the species.

Polyols as used herein can include but are not limited to straight-chain molecules having carbon chain length of about 3 to about 8, specifically, about 5 to about 6, with the number of hydroxyl groups of about 3 to about 8, specifically, about 5 to about 6. “Polyols” as described herein are understood to be substances with 3 or more hydroxyl groups (i.e., including triols but excluding diols). For example, straight chain polyols can include erythritol, glycerol, mannitol, sorbitol, xylitol, and combinations comprising at least one of the foregoing. The polyols can be naturally or synthetically derived, and can also include cyclic, or combinations of straight chain and cyclic structures. For example, isomalt, lacitol, maltitol, and various HSH's (hydrogenated starch hydrosylates) can be used. For example, mannitol or sorbitol (both C6H14O6) or combinations of the two can be used. Polyols that contain, in addition to the desired minimum (3) hydroxyl groups, other functional groups in the molecule such as aldehydes, ketones, carboxylate, thiols, etc. can also be used in the hair styling products disclosed herein. For example, methyl 2,5,6-trihydroxyhexanoate, or other functionalized polyols can find use herein. Additionally, natural sugar monosaccharides that are in equilibrium mixture of open straight chain form and cyclic aldol or ketol form can also be used. Many small organic molecules, or mixtures thereof, can also be used herein to provide hard-hold provided they minimally have three or more hydroxyl groups. The polyol or polyols can be incorporated in the compositions and/or hair styling products disclosed herein in amounts of about 1 wt. % to about 20 wt. % by weight in the composition, and preferably about 5% wt. % to about 10 wt %. It can also be desirable to maintain a ratio of polyol to total synthetic and natural polymers of about 1:3 to about 1:10. Further the mass ratio of polyol to total water content of the solution varies from about 2:1 to about 1:5, preferably from about 1:1 to about 1:2. While the use of a single polyol may be preferable for economic reasons, nothing in this disclosure limits the polyol to a single species. Mixtures of polyols have the advantage of additional degrees of freedom to the solution allowing a wider range of properties with corresponding overhead costs as discussed above.

The polymer or copolymer used in the hair styling products disclosed herein can be nonionic, anionic, zwitterionic or amphoteric or cationic synthetic homopolymers, and copolymers. For example, nonionic polymers can include polyvinylpyrrolidone (PVP), copolymers of N-vinylpyrrolidone and vinyl acetate, and/or vinyl propionate, polyvinylcaprolactam, polyvinylamides and salts thereof, and copolymers of vinylpyrrolidone and dimethylaminoethyl methacrylate, copolymers of vinylpyrrolidone (VP) and dimethylamino propylacrylamide (DMAPA), terpolymers of vinylcaprolactam, vinylpyrrolidone and dimethylaminoethyl methacrylate, polysiloxanes, and combinations comprising at least one of the foregoing.

Anionic polymers include vinyl acetate/crotonic acid, vinyl acetate/acrylate and/or vinyl acetate/vinyl neodecanoate/crotonic acid copolymers, sodium acrylate/vinyl alcohol copolymers, sodium polystyrenesulfonate, ethyl acrylate/-N-tert-butylacrylamide/acrylic acid copolymers, vinylpyrrolidone/vinylacetate/itaconic acid copolymers, acrylic acid/acrylamide copolymers and/or sodium salts thereof, homo-and/or copolymers of methacrylic acid and/or salts thereof, and acrylate/hydroxyacrylate, octylacrylamide/acrylate or methacrylate and/or butyl acrylate/N-vinylpyrrolidone copolymers or polystyrenesulfonates.

Amphoteric polymers include copolymers of N-octylacrylamide, methacrylic acid and tert-butylaminoethyl methacrylate of the “amphomer” type, copolymers of methacryloylethylbetaine an alkylmethacrylates of the “yukaformer” type, copolymers of monomers containing carboxyl groups or sulfone groups, for example methacrylic acid and itaconic acid, with basic group-containing monomers such as mono- or dialkylaminoalkyl methacrylates and/or mono- and dialkylaminoalkyl methacrylamides, copolymers of N-octylacrylamide, methyl methacrylate, hydroxypriopyl methacylate, N-tert-butylaminoethyl methacrylate and acrylic acid.

Cationic polymers include vinylpyrrolidone/vinylimidazolium methochloride copolymers, quaternized vinylpyrrolidone/diakylaminoalkyl methacrylate copolymers, cationic cellulose derivatives, such as hydroxyethylcellulose/dimethylalkylammonium chloride copolymers, and terpolymers of vinylcaprolactam/vinylpyrrolidone with dimethylaminoethyl methacrylate or vinylimidazolium methochloride and acrylamido copolymers.

That being said, the hair styling products disclosed herein can comprise compositions comprising vinyl pyrrolidone/dimethylaminoethyl methacrylates copolymer, vinyl acetate/crotonates/vinyl neodecanoate copolymer, octyl acrylamide/acrylates/butylaminoethyl methacrylate copolymer, vinyl acetate/crotonates, polyvinylpyrrolidone (PVP), polyvinylpyrrolidone/vinyl acetate copolymer, PVP acrylates copolymer, VP/DMAPA acrylates copolymer, vinyl caprolactam, vinyl acetate/crotonic acid/vinyl proprionate, acrylates/acrylamide, acrylates/octylacrylamide, acrylates copolymer, acrylates/hydroxyacrylates copolymer, and alkyl esters of polyvinylmethylether/maleic anhydride, diglycol/cyclohexanedimethanol/isophthalates/sulfoisophthalates copolymer, vinyl acetate/butyl maleate and isobornyl acrylate copolymer, vinylcaprolactam/PVP/dimethylamino ethyl methacrylate, vinyl acetate/alkylmaleate half ester/N-substituted acrylamide terpolymers, vinyl caprolactam/vinylpyrrolidone/dimethylaminoethyl methacrylates copolymer, vinyl caprolactam/vinylpyrrolidone/methacryloamidopropyl trimethylammonium chloride terpolymer, methacrylates/acrylates copolymer/amine salt, polyvinylcaprolactam, polyurethanes, polyquaternium-4, polyquaternium-10, polyquaternium-11, polyquaternium-46, hydroxypropyl guar, hydroxypropyl guar hydroxypropyl trimmonium chloride, polyvinyl formamide, polyquaternium-7, and hydroxypropyl trimmonium chloride guar.

The polymers for use in the hair styling products and compositions disclosed herein can include in various ratios and amounts of polyvinylpyrrolidone, polyvinylpyrrolidone/vinyl acetate copolymer, vinyl pyrrolidone/dimethylaminoethyl methacrylates copolymer, and vinyl caprolactam/vinyl pyrrolidone/dimethylaminoethyl methacrylates copolymer. The preferred total amount of synthetic fixative polymers is about 30 wt. % to about 70 wt. %, with either one of these or combinations thereof. In order to keep the VOC level in the composition at or below 2%, it is important to use polymers that are available in aqueous or nearly aqueous solution, or in powdered faun, rather than using polymers supplied in solvent solutions that will contribute to the VOC content of the final composition. The polyvinylpyrrolidone polymer can be present in an amount of 1.2 weight percent (wt. %) to 15 wt. %, specifically, 2 wt. % to 8 wt. %, and even more specifically, 5 wt. % to 7 wt. %. The vinylpyrrolidone/DMAPA acrylates copolymer can be present in an amount of 0.3 wt. % to 5 wt. %, specifically, 0.5 wt. % to 3 wt. %, and more specifically, 0.8 wt. % to 1.3 wt. %. The vinyl caprolactam/vinyl pyrrolidone/dimethylaminoethyl methacrylate copolymer can be present in an amount of 1 wt. % to 10 wt. %, specifically, 2 wt. to 8 wt. %, and more specifically 3 to 5 wt. %. Use of these polymers in the amounts described can provide a surprising synergy resulting a hair styling composition having increased hold attributes as compared to a hair styling composition comprising different or an increased number of polymers.

For example, the compositions can include polyvinylpyrrolidone 20% aqueous solution, or polyvinylpyrrolidone powder, sold under the trade names of Luviskol K-90® and Luviskol-PVP® respectively commercially available from BASF (both 0% VOC contribution), vinyl pyrrolidone/dimethylaminoethyl propylacrylamide acrylates copolymer (VP/DMAPA acrylates copolymer-copolymer), sold under the trade name of Styleze CC-10® commercially available from ISP (0% VOC contribution), and vinyl caprolactam/vinyl pyrrolidone/dimethylaminoethyl methacrylate copolymer with 0.77% ethanol, sold under the trade name of Advantage®-S Solution from ISP (0.77% VOC contribution).

The compositions disclosed herein can also include starch, modified starches and/or other cellulosic material or combinations of these, for modifying the quality of the dried film on the hair and for modifying the viscosity of the actual composition. For example, of use in the compositions disclosed herein include celluloses, cellulose derivatives, cellulose gums, ethoxylated celluloses, starch or gums, guar gum, guar hydroxypropyl trimonium chloride, xanthan gum, hydroxypropyl guar, karaya gum, as well as combinations comprising at least one of the foregoing. Also of use in the compositions disclosed herein are pregelatinized crosslinked starch derivatives, including hydroxypropyl distarch phosphate, as described in U.S. Patent Application Publication US2005/0191264 and incorporated herein by reference. Cellulosic materials for use herein can include nonionic or cationic saccharides such as cellulose ethers including methyl cellulose, carboxymethyl cellulose, hydroxy propyl methyl cellulose, hydroxyethyl cellulose, hydroxypropyl cellulose, and ethyl hydroxyethyl cellulose, dextrans obtained from Sigma, Kitamer PC, a chitosan carboxylate and Kytamer L, a chitosan lactate obtained from Amerchol, Gafquat HS-100, Polyquatemium-28 from International Specialties, polyquaternium4, polyquaternium-10, sodium alginate, agarose, amylopectins, amyloses, arabinans, arabinogalactans, arabinoxylans, carrageenans, gum arabic, cellulose derivatives such as methylcellulose, hydroxypropylmethylcellulose, hydroxyethyl cellulose, carboxymethylcellulose, carboxymethylguar gum, carboxymethyl(hydroxypropyl)guar gum, hydroxyethylguar gum, hydroxypropylguar gum, cationic guar gum, chondroitins, chitins, chitosans, cocodimonium hydroxypropyl oxyethyl cellulose, colominic acid [poly(N-acetyl-neuraminic acid], corn starch, curdlan, dermatin sulfate, furcellarans, dextrans, cross- linked dextrans known as dextranomer (Debrisan), dextrin, emulsan, flaxseed saccharide (acidic), galactoglucomannans, galactomannans, glucomannans, glycogens, guar gum, or hydroxyethylstarch, hydroxypropylstarch, hydroxypropylated guar gums, gellan gum, glucomannans, gellan, gum ghatti, gum karaya, gum tragacanth (tragacanthin), heparin, hyaluronic acid, inulin, keratan sulfate, konjac mannan, laminarans, laurdimonium hydroxypropyl oxyethyl cellulose, liposan, locust bean gum, mannans, nigeran, nonoxylnyl hydroxyethyl cellulose, okra gum, oxidized starch, pectic acids, pectins, polydextrose, potato starch, protopectins, psyllium seed gum, pullulan, sodium hyaluronate, steardimonium hydroxyethyl cellulose, raffinose, rhamsan, tapioca starch, welan, levan, scleroglucan, stachyose, succinoglycan, wheat starch, xanthan gum, xylans, xyloglucans, and mixtures thereof. Microbial saccharides can be found in the fourth edition of Kirk-Othmer Encyclopedia of Chemical Technology, Fourth Edition. Vol. 16, John Wiley and Sons, NY pp. 578-611, 1994. Complex carbohydrates can be found in the fourth edition of Kirk-Othmer Encyclopedia of Chemical Technology, Fourth Edition. Vol. 4, John Wiley and Sons, NY pp. 930-948, 1994. For example, hydroxyethylcellulose and a modified corn starch from National Starch found under the trade name Amaze® can be used. The compositions disclosed herein can include about 0.01 wt. % to about 10 wt. % total cellulosic and starch materials, by weight in the composition. For example, hydroxyethylcellulose and Amaze® modified corn starch from National Starch can be used, together in the composition at a total of about 0.1 wt. % to about 5 wt. % by weight, and preferably 0.3 to 1.2 wt %.

The compositions can also include a thickening polymer, such as a high molecular weight polyacrylate. Thickeners can be utilized alone or in combination so long as the chosen thickeners are compatible with the hair styling composition. Thickeners may include, but are not limited to, acrylic acid homopolymers under the Carbopol® trade name from BF Goodrich, acrylates/C10-30 alkyl acrylate crosspolymer (Carbopol® 1342, 1382, Pemulins® TR-1 and TR-2 from BF Goodrich), Acrylates/Steareth-20 Itaconate Copolymer, Acrylates/Ceteth-20 Itaconate Copolymer from National Starch, Bentonite, PVM/MA Decadiene Crosspolymer from International Specialties Products, Acrylates/steareth-20 methacrylate copolymer, Acrysol® ICS-1 from Rohm and Haas Co., acrylamide/sodium acrylate copolymer, Hostacerin® PN 73, Hoecsht A G., acrylate copolymer (Antil® 208) supplied by Degussa-Goldschmidt, acrylic acid/acrylonitrogens copolymer (Hypan® SA-100H, SR-150H) supplied by Lipo, Acrylic/acrylate copolymer (Carboset® 514, 515, 525, XL-19, XL-19X2, XI-28, XL40, 526) supplied by BF Goodrich, Ammonium acrylateslacrylonitrogens copolymer (Hypan® SS-201) from Lipo, Quatemium-18 Bentonite, Sodium salt of crosslinked poly(acrylic acid) under the tradenames PNC® 430, PNC® 410, PNC® 400 from 3V, Stearalkonium Bentonite, Claytone, supplied by Southern Clay, Quatemium-18 Hectorite (Bentone 38), Stearalkonium Hectorite (Bentone 27) supplied by Rheox, acrylamide/sodium acrylate copolymer (Hostacerin PN 73) supplied by Hoechst, Poly(acrylic acid) known as Carbopol® 400 series (BF Goodrich) or Aquatreat® (Alco 3V), polyquaternium-18 (Mirapol® AZ-1) from Rhone Poulenc, polyquaternium-27, polyquaternium-31, polyquaternium-37, trihydroxystearin (Thixcin from Rheox; Flowtone from Southern Clay), Dimethylaminoethyl methacrylamide and acrylamide copolymer (Salcare® SC63 from Ciba Specialties), Acrylic polymer cationic thickening agents (Synthalen® CR and its related compounds) from 3V Sigma. Other thickeners and polymers can be found in the “The Encyclopedia of Polymers and Thickeners for Cosmetics,” Cosmetics and Toiletries, Lochhead, R., pp. 95-138, Vol. 108, (May 1993). Thickeners, when present, can be incorporated in amounts of about 0.01 wt. % to about 5 wt. % by weight active polymer.

The compositions can also include petrolatum or other waxes and oils. Petrolatum is a mixture of hydrocarbons that finds use in various personal care products. The petrolatum used in the compositions disclosed herein can include white petrolatum USP, petrolatum USP, mineral jelly, and ointment base. The melting point ranges of the preferred petrolatum for use in the present invention can be about 80° F. to about 135° F. For example, UltraPure® Liquid Petrolatum USP from Ultra Chemicals that has a melting point range of about 105° -115° F., or various grades (ranging in color) of Penreco® Petrolatum USP having melting point ranges around 122° -135° F. can be used. The compositions can also contain an emulsifying wax and/or oil. Such materials include the non-limiting examples of bees' wax, candelilla wax, carnauba wax, emulsifying wax (for example Polawax® from Croda) and Jojoba, safflower, canola (tribehenin), tallow, lard, palm, castor, sunflower seed, or soya bean oil oils, or hydrogenated derivatives thereof. Most preferred is to incorporate Polawax®, jojoba oil, safflower oil, tribehenin, and/or hydrogenated castor oil, singularly, or in any combination. When desired in the present compositions, any combination of these materials can be used in an amount of about 0.1 wt. % to about 5 wt. % by weight of the total composition. For example, petrolatum, wax, and/or oil in combination or individually, can be used in an amount of about 1 wt. % to about 3 wt. % by weight in the composition.

Fatty alcohols that may find use in the compositions disclosed herein can include naturally derived and synthetic materials. These are high molecular weight straight or branched chain primary alcohols. For example, the fatty alcohol can be lauryl (C12), myristyl (C14), cetyl or palmityl (C16), stearyl (C18), oleyl (C18-unsaturated) and linoleyl (C18-polyunsaturated) alcohols, or combinations thereof. Ceteryl alcohol can also be used and is a mixture of cetyl and stearyl alcohols. The fatty alcohol, when present, can be incorporated in an amount of about 0.01 wt. % to about 5 wt. % by weight in the composition.

Also of use as an optional ingredient in the compositions disclosed herein are emulsifiers. Emulsifiers for use in cosmetic applications are amply listed in McCutcheon's Emulsifiers and Detergents. Many emulsifiers are nonionic esters or ethers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditol as hydrophilic moiety. The hydrophilic moiety can contain polyoxypropylene. The emulsifiers additionally contain a hydrophobic alkyl, alkenyl, or arylalkyl moiety, normally containing from about 8 to about 50 carbons. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil, sunflower seed oil, or soya bean oil. Such nonionic surfactants can also be derived from a polyhydroxy compound such as glycerol or sorbitol or other alditols. Examples of such emulsifiers include ceteareth-10 to -25, ceteth-10-25, steareth-10-25 (i.e. C16 to C18 alcohols ethoxylated with 10 to 25 ethylene oxide residues) and PEG-15-25 stearate or distearate. Other suitable examples include C10-C20 fatty acid mono, di- or tri-glycerides. Further examples include C18-C22 fatty alcohol ethers of polyethylene oxides (8- to 12-EO). Other examples of useful emulsifiers are fatty acid mono or possibly diesters of polyhydric alcohols such as glycerol, sorbitol, erythritol, or trimethylolpropane. The fatty acyl moiety is often from C14 to C22 and is saturated in many instances, including cetyl, stearyl, arachidyl, and behenyl. Examples include monoglycerides of palmitic or stearic acid, sorbitol mono or diesters of myristic, palmitic, or stearic acid, and trimethylolpropane monoesters of stearic acid. Another usable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers often terminate in C1 to C12 alkyl groups. Such emulsifiers and co-emulsifiers are widely available under many trade names and designations including Abil®, Arlacel®, Brij®, Cremophor®, Dehydrol®, Dehymuls®, Emerest®, Lameform®, Pluronic®, Prisorine®, Quest PGPR®, Span® Tween®, SF1228, DC3225C and Q2-5200.

Desirable emulsifiers can include any combination of fatty alcohols (such as mentioned previously), phosphate-based emulsifying waxes, sorbitan monooleates and stearates and other carbohydrate esters of fatty alcohols and their ethoxylated derivatives, and the polyalkylene glycols and polyethoxylated waxes. For example, a fatty alcohol blend for optional use in the present compositions is Crodafos® CES (white solid or flakes) from Croda, which is a blend of ceteryl alcohol, dicetyl phosphate and ceteth-10 phosphate. Apifil PEG-8 beeswax emulsifier, which is a combination of fatty acid esters and polyethylene glycol and is a nonionic self-emulsifying base can also be used. When desired in the composition, the emulsifier can be present in an amount of about 0.01 wt. % to about 10 wt. % by weight in the composition and preferably 0.2 to 0.8 wt. %.

In the set of formulations disclosed herein, emulsifiers have shown a dramatic and unexpectedly large impact on the viscosity and stability of the viscosity over time. Indeed, a change in 1 weight % (wt. %) of the emulsifier produced a change in viscosity of approximately 160,000 cSt. This was true over a variety of compositions. While it is not completely clear why this is the case, emulsifiers and known for their ability to interact with both hydrophilic and hydrophobic materials. Emulsifiers may allow the formulation of micelles to sequester a minority material from the bulk of the solution. It seems likely that the emulsifier is adjusting the availability of water to lubricate the polyols. Regardless, the large impact of the emulsifier concentration allows tuning of the viscosity over a large range with little change to overall the composition of the formulation. This provides significant benefits when a formulation has been identified with acceptable product performance but there is a desire to thicken the product without significant adjustment to the composition, such as might be required by adding a quantity of long chain polymer or volatile small molecule. Further, because of their ability to stabilize surfaces and phase interfaces, emulsifiers may impact the evaporation rate or drying time of a formulation.

Also, the compositions disclosed herein can include “oil-soluble film former” polymers. Non-limiting examples of the oil-soluble film former include polymethylsilsesquioxanes; acrylic fluorinated emulsion film formers, such as Foraperle® film formers (e.g., Foraperle® 303 D available from Elf Atochem); GANEX® copolymers, such as butylated PVP, PVP/Hexadecene copolymers, PVP/Eicosene copolymers, and tricontanyl; Poly-(vinylpyrrolidone/diethylaminoethyl methacrylate) copolymers and PVP/Dimethylaminoethylmethacrylate copolymers such as Copolymer 845 available from I.S.P.; Resin ACO-5014 (Imidized IB/MA copolymer); other PVP based polymers and copolymers; silicone gums; cyclomethicone copolymers and dimethicone crosspolymers, such as Dow Corning® 2-9040 and those disclosed in U.S. Pat. No. 5,654,362, the disclosure of which is hereby incorporated by reference; trimethyl siloxysilicates such as SR 1000, 554230, and SS4267 available from GE Silicones; alkyl cycloalkylacrylate copolymers, such as those disclosed in WO 98/42298, the disclosure of which is hereby incorporated by reference; Mexomere® film formers and other allyl stearate/vinyl acetate copolymers; polyolprepolymers, such as PPG-12/SMDI copolymer, also called Poly-(oxy-1,2-ethanediyl), α-hydro-ω-hydroxy-polymer with 1,1′-methylene-bis-(4-isocyanatocyclohexane) available from Barnet; and Avalure® AC Polymers (Acrylates Copolymer) and Avalure® UR polymers (Polyurethane Dispersions), available from BFGoodrich. Preferred for use in the present invention is the already mentioned tricontanyl PVP copolymer sold under the trade name Ganex® WWP-660 from ISP. Any combination of the Ganex® V/WP grades can be used in the compositions disclosed herein and provide water and wear resistance and a moisture barrier to the set hair. The oil-soluble film former polymer can be present in the composition in an amount of about 0.1 wt. % to about 5 wt. % by weight.

Also optional to the compositions disclosed herein is the addition of a chelant. Chelants that can find use herein include but are not limited to phosphates (organic and inorganic), NTA, the various ethylenediaminetetraacetic acid (EDTA) derivatives, and lower molecular weight polyacrylates. For example, the present invention may include trisodium or tetrasodium EDTA, various salts of NTA, phosphate esters, or Acusol® 445 from Rohm and Haas. The chelant can be present in the composition in an amount of about 0.001 wt. % to about 1 wt. % by weight.

Combining constituents that increased the viscosity in proportion to constituents the decreased the viscosity, facilitates forming a solution where the overall rate of change of the viscosity of the solution over time low.

Composition Table for Figures.

TABLE 1
Compositions of Runs, rounded to 0.1 wt. %

| | | | | | | Alf | Alf | | | | |
|---|---|---|---|---|---|---|---|---|---|---|---|
| Run | Luv | Adv | Sty | Nat | Ama | 16 | 18 | Ste | Bee | Cro | Other |
| 1 | 34.4 | 11.2 | 10.0 | 0.5 | 1.0 | 1.5 | 1.5 | 1.5 | 0.5 | 2.0 | 23.3 |
| 2 | 15.0 | 9.0 | 13.0 | 0.0 | 1.0 | 1.5 | 0.2 | 1.5 | 0.1 | 1.0 | 45.0 |
| 3 | 45.0 | 15.0 | 7.0 | 0.5 | 1.0 | 1.5 | 0.2 | 1.5 | 0.5 | 2.0 | 13.1 |
| 4 | 45.0 | 9.0 | 7.0 | 0.0 | 1.0 | 0.2 | 1.5 | 0.2 | 0.5 | 1.0 | 21.9 |
| 5 | 45.0 | 15.0 | 7.0 | 0.0 | 0.1 | 0.2 | 0.2 | 0.2 | 0.1 | 1.0 | 18.4 |
| 6 | 15.0 | 15.0 | 13.0 | 0.0 | 0.1 | 1.5 | 1.5 | 1.5 | 0.5 | 1.0 | 38.3 |
| 7 | 15.0 | 9.0 | 7.0 | 0.0 | 0.1 | 0.2 | 0.2 | 1.5 | 0.5 | 2.0 | 51.8 |
| 8 | 17.5 | 15.0 | 7.0 | 0.3 | 1.0 | 0.2 | 0.2 | 0.2 | 0.5 | 1.5 | 43.9 |
| 9 | 30.0 | 12.0 | 10.0 | 0.3 | 0.5 | 0.9 | 0.9 | 0.9 | 0.3 | 1.5 | 30.1 |
| 10 | 15.0 | 9.0 | 7.0 | 0.0 | 0.1 | 0.2 | 0.2 | 1.5 | 0.5 | 2.0 | 51.8 |
| 11 | 45.0 | 9.0 | 13.0 | 0.5 | 0.1 | 0.2 | 1.5 | 1.5 | 0.1 | 2.0 | 14.4 |
| 12 | 36.6 | 15.0 | 13.0 | 0.4 | 1.0 | 0.2 | 1.5 | 0.6 | 0.5 | 1.7 | 16.8 |
| 13 | 15.0 | 9.0 | 7.0 | 0.5 | 0.1 | 1.5 | 1.5 | 0.2 | 0.1 | 2.0 | 50.4 |
| 14 | 15.0 | 15.0 | 13.0 | 0.0 | 1.0 | 0.2 | 1.5 | 0.2 | 0.1 | 2.0 | 39.3 |
| 15 | 45.0 | 9.0 | 7.0 | 0.0 | 1.0 | 0.2 | 1.5 | 0.2 | 0.5 | 1.0 | 21.9 |
| 16 | 15.0 | 9.0 | 7.0 | 0.5 | 0.1 | 1.5 | 1.5 | 0.2 | 0.1 | 2.0 | 50.4 |
| 17 | 45.0 | 9.0 | 13.0 | 0.0 | 0.1 | 1.5 | 0.2 | 0.2 | 0.5 | 2.0 | 15.8 |
| 18 | 30.0 | 12.0 | 10.0 | 0.3 | 0.5 | 0.9 | 0.9 | 0.9 | 0.3 | 1.5 | 30.1 |
| 19 | 25.4 | 9.0 | 8.9 | 0.5 | 1.0 | 0.2 | 1.4 | 1.5 | 0.1 | 1.0 | 38.3 |
| 20 | 15.0 | 9.0 | 13.0 | 0.0 | 1.0 | 1.5 | 0.2 | 1.5 | 0.1 | 1.0 | 45.0 |
| 21 | 15.0 | 15.0 | 13.0 | 0.0 | 0.1 | 1.5 | 1.5 | 1.5 | 0.5 | 1.0 | 38.3 |
| 22 | 15.0 | 9.0 | 13.0 | 0.5 | 0.1 | 0.2 | 0.2 | 0.2 | 0.5 | 1.0 | 47.5 |
| 23 | 45.0 | 15.0 | 13.0 | 0.5 | 1.0 | 1.5 | 1.5 | 1.5 | 0.5 | 2.0 | 5.8 |
| 24 | 30.0 | 12.0 | 10.0 | 0.3 | 1.0 | 0.9 | 1.5 | 1.5 | 0.1 | 1.5 | 28.6 |
| 25 | 17.5 | 15.0 | 7.0 | 0.3 | 1.0 | 0.2 | 1.5 | 0.3 | 0.1 | 1.5 | 43.0 |
| 26 | 45.0 | 15.0 | 10.5 | 0.2 | 1.0 | 1.5 | 0.3 | 1.5 | 0.1 | 1.0 | 11.2 |
| 27 | 45.0 | 14.9 | 10.1 | 0.0 | 1.0 | 0.3 | 1.5 | 0.3 | 0.1 | 1.5 | 12.7 |
| 28 | 30.0 | 9.9 | 7.0 | 0.0 | 1.0 | 1.3 | 0.3 | 1.5 | 0.1 | 1.5 | 34.9 |
| 29 | 39.0 | 14.2 | 8.9 | 0.5 | 1.0 | 1.5 | 1.5 | 0.3 | 0.1 | 1.1 | 19.2 |
| 30 | 30.0 | 11.2 | 13.0 | 0.5 | 1.0 | 1.5 | 0.3 | 0.3 | 0.1 | 1.0 | 28.5 |
| 31 | 41.4 | 15.0 | 9.9 | 0.0 | 1.0 | 1.5 | 0.3 | 1.5 | 0.1 | 1.0 | 15.7 |
| 32 | 15.0 | 9.0 | 11.7 | 0.4 | 1.0 | 0.7 | 1.5 | 1.5 | 0.1 | 1.8 | 44.7 |
| 33 | 42.8 | 15.0 | 13.0 | 0.0 | 1.0 | 0.8 | 0.3 | 0.3 | 0.1 | 1.0 | 13.2 |
| 34 | 15.0 | 9.1 | 7.0 | 0.1 | 1.0 | 1.0 | 0.3 | 0.3 | 0.1 | 1.0 | 52.5 |
| 35 | 30.0 | 10.3 | 12.9 | 0.1 | 1.0 | 0.2 | 1.5 | 0.3 | 0.1 | 1.0 | 29.9 |
| 36 | 27.1 | 9.0 | 12.4 | 0.2 | 1.0 | 0.2 | 1.5 | 1.5 | 0.1 | 1.7 | 32.6 |
| 37 | 34.8 | 15.0 | 7.0 | 0.2 | 1.0 | 0.6 | 0.3 | 0.3 | 0.1 | 2.0 | 26.2 |
| 38 | 45.0 | 15.0 | 9.4 | 0.5 | 1.0 | 0.2 | 1.5 | 1.5 | 0.1 | 1.0 | 12.1 |
| 39 | 34.8 | 15.0 | 10.6 | 0.1 | 1.0 | 0.3 | 1.5 | 1.5 | 0.1 | 1.6 | 20.7 |
| 40 | 15.0 | 9.0 | 11.7 | 0.4 | 1.0 | 0.7 | 0.3 | 1.5 | 0.1 | 1.8 | 46.0 |
| A | 25.0 | 9.0 | 11.0 | 0.0 | 1.0 | 0.3 | 0.3 | 1.5 | 0.1 | 1.0 | 38.3 |
| B | 17.0 | 15.0 | 11.0 | 0.0 | 1.0 | 0.3 | 0.3 | 1.5 | 0.1 | 1.0 | 40.3 |

Column identifications: Run is Run number. Luv = Luviskol K 90 - 20%, Adv = Advantage S E Solution, Sty = Styleze CC-10, Nat = Natrosol 250 HR, Ama = Amaze; Alf 16 = Alfol 16 NF, Alf 18 = Alfol 18 alcohol, Ste = Steareth-21, Bee = Beeswax, Cro = Crodafos CES, Other includes: Water, Petrolatum, Sorbitol, Perfume, and Preservative. Values are weight % of the resulting solution.

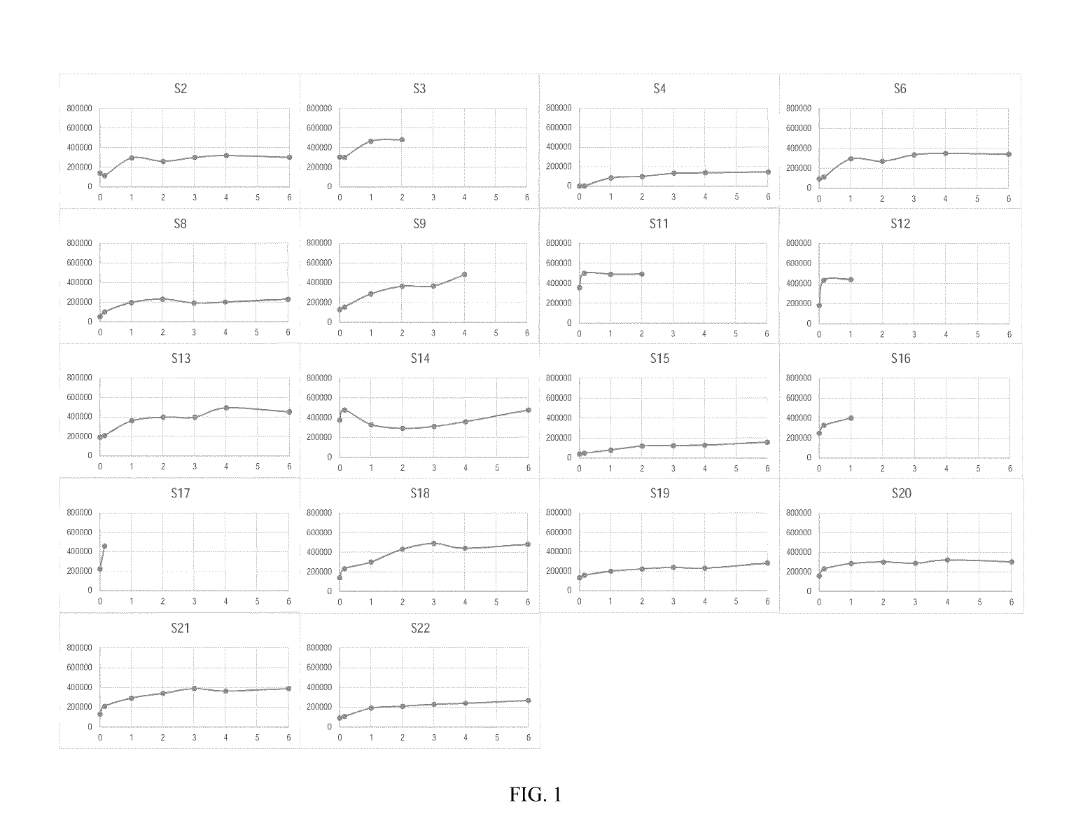

FIG. 1 shows the time response graphs for viscosity of runs 2-6, 8-9, and 11-22. Most show a notable increase in viscosity in the first week. Some stabilize while other continue to increase in viscosity. In the graphs, the vertical axes are cStokes (cSt) and the horizontal axes are weeks. Viscosity was measured at 10 RPM using a Brookfield T Bar spindle Size E in an open 16 oz (480 ml) glass jar. When the measured viscosities is above 350,000 cSt (cP) are become difficult to dispense from some container designs.

The viscosity graphs were grouped by shape to separate “good” profiles such as s19 from “unacceptable” profiles such as s11 or s9. Performing a t-Test (Excel, 2-tailed, equal variance) indicated that Luviskol 90 and Others were significant at 0.05 and Crodafos CES was significant at 7.0×10̂−6 for determining the category of viscosity response. The other components were not significant at a p<0.05 level.

In the test matrix, Luviskol 90 had a significant Pearson's correlation (Excel, Correl) of −0.96 with Other indicating some confounding. This is likely do the much larger ranges of Luviskol considered compared with the other components. This correlation coefficient represented a p value of <0.01. Amaze and Beeswax were negatively correlated at a 0.05 significance level. Luviskol 90 and Advantage were positively correlated at a 0.05 significance level. No other significant correlations were found in the composition data.

Analyzing the results of the various components on a viscosity contribution per weight percent provides the following numbers:

TABLE 2
Contributions of various components in change in cSt per 1 wt. %. Data
rounded to two significant figures.

| | Estimated | Mean | | More of |
|---|---|---|---|---|
| | Change in | high wt. | Mean change | Component |
| | Viscosity (cSt) | %-mean low | in Viscosity | Causes |
| Component | per 1 wt. % | wt. % | (cSt) | Viscosity to: |
| Luv & Adv | −7,900 | 28 & 5.8 | −270,000 | Decrease |
| Sty | 19,000 | 5.2 | 100,000 | Increase |
| Nat | 210,000 | 0.47 | 100,000 | Increase |
| Alf 16 | 83,000 | 1.2 | 100,000 | Increase |
| Cro | 160,000 | 0.92 | 150,000 | Increase |
| Other | −22,000 | 29 | −650,000 | Decrease |

Note that the similarity of magnitude of absolute viscosity change regardless of the approximately two orders of magnitude weight percentage change This may indicate some confounding in the test matrix. However, it is clear that water concentration (other) is a major contributor. This is consistent with the model proposed above where water, especially free water concentration controlled by the interaction with a small polar molecule to provided much of the mobility in the solution. Note the very strong impact of Nat and Cro on the viscosity per wt %. Natrosol is noted for its strong interaction with ionic polymers. (Source: Natrosol Family Product Literature).

The graphs in FIG. 1 show a number of different behaviors, some of the graphs show stabilization in the 2-3 week range while others continue to increase over time. Graph s14 in FIG. 1 shows a decrease in viscosity over time. Looking at the trending after the 2nd week using the methodologies described above did not identify any particular component to be associated with instability of the viscosity in the formulations. As a result, the long term stability is like the result of interactions between the various components. As some of the components are associated with less and others greater viscosity, it seems that the long term viscosity stability is dependent on balancing the contributions of the various components. This is consistent with the reversal of viscosity, for instance seen in s17.

Changes in viscosity can be detected over even relatively short time frames such as shown in the graphs. Since the changes in solution properties appear to be thermodynamically driven and not chemical reactions, they can be expected to be slow and dampen over time as the solution approaches equilibrium. This is evident in the graphs where many of the solutions show decreasing slope over time. Thus, short term response may provide indications about long term stability of the solution. However, the presence of graphs that trend in one direction and then reverse, such as s 17 indicate that there may multiple contributions to viscosity operating on different time scales.

The following method provides teaching as to how to apply the described discovery. Determine a target formulation with acceptable short term properties. Measure the impact of controlled amounts of emulsifier and use that information to tune the overall viscosity of the solution. Emulsifiers in these polymer based solutions provide an unexpectedly strong impact on the viscosity of the solutions.

The following formulations take advantage of the insights gathered from the work described in Table 1. Formula C has a tendency to increase in viscosity making it difficult to dispense from containers. Formula D uses reduces amount of Natrosol 250 HR and Crodafos CES and increased amounts of Advantage E S Solution to produce a more stable solution less prone to viscosity changes during aging.

TABLE 3
Formulations

| | Formula C | Formula D |
|---|---|---|
| Component | Weight % | Weight % |
| Water, demineralized | 30.50 | 30.70 |
| Luviskol K 90-20%* | 30.00 | 30.00 |
| Advantage S Solution** | 12.00 | 13.65 |
| Sorbitol 70% HS DAB*** | 10.00 | 10.00 |
| Styleze CC-10**** | 10.00 | 9.00 |
| Natrosol 250 HR | 0.30 | 0.10 |
| Amaze | 0.50 | 0.50 |
| Perfecta Petrolatum | 2.00 | 2.00 |
| Crodafos CES | 1.50 | 1.25 |
| Alfol 16 NF | 0.75 | 0.50 |
| Alfol 18 Alcohol | 0.75 | 0.75 |
| Steareth-21 | 0.75 | 0.75 |
| Bees wax | 0.30 | 0.15 |
| Perfume | 0.35 | 0.35 |
| Glydant***** | 0.30 | 0.30 |

*20 wt. % Polyvinylpryillidone, 0.005 wt. % Polyaminopropyl Biguanide, HCl, 80 wt. % water.
**30 wt. % Vinyl Caprolactam/VP/Dimethylaminoethyl Methacrylate Copolymer, 0.45 wt. % 1,2propane diol, 0.22 wt. % Diazolidinyl urea, 0.0025 wt. % Iodo-2-propynyl butylcarbamate, 3, 69.3275 wt. % water.
***70 wt. % Sorbitol, 30 wt. % water.
****10 wt. % VP/DMAPA Acrylates copolymer, 0.10 wt. % Alkyldimethylbenzylammonium chloride, 90% water.
*****55 wt. % Dimethylol-5,5-dimethylhydantoin, 1,3, 45 wt. % water.

The compositional differences between these two formulas is small. Water is 0.2 wt. % different. Advantage S solution is 1.65 wt. % different. Styleze CC-10 is 1 wt. % different. Natrosol 250 HR is 0.2 wt. % different. Crodafos CES is 0.25 wt. % difference. Alfol is 0.25 wt. % difference. Bees wax is 0.15 wt. % difference. Accordingly, one of ordinary skill in the art would expect similar performance out of the two formulations. And indeed, short term or as mixed basis the two formulas exhibit similar behavior. However, surprisingly, the two formulas show significantly different shelf life behavior which results in formula D is shelf stable while formula C becomes increasingly difficult to dispense and use due to its increasing viscosity. Formula D was developed using Formula C, the viscosity data described above in FIG. 1, and the method described above. The unexpectedly strong impact of the emulsion agent Crodafos in isolating water from the solution was a significant change that impacted the long term stability of the viscosity. The interaction of Natrosol 250 HR with the ionic polymers has a strong impact on viscosity.

The formulations are low VOC (volatile organic compound) formulation with less than 2% of VOCs in order to comply with certain regulatory requirements, the formulations do not include significant amounts of volatile components such as low weight alcohols. Instead, the primary solvent is water.

Several of the components increased the viscosity of the solution. Specifically, increased concentrations of Crodafos CES, Natrosol 250 HR, Alfol 16 NF, and Styleze CC-10 were with increased the viscosity of the solution. Other components, specifically Luviskol K 90 -20%, Advantage S E Solution, and water correlated with decreasing viscosity of the solutions. Balancing these changes over time produces a solution with greater viscosity stability. Viscosity for these experiments was measured using a TE spindle in glass 16 oz. jar at 10 RPM. Due to the high viscosities involved, the use of a rotational measurement method is preferred for measuring viscosity of the solutions. For developing thinner products other measurement methods may also be used.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair styling composition, comprising:
a combination of three polymers, comprising a total of 4 wt. % to 25 wt. %, the three polymers comprising:
1.2 wt. % to 15 wt. % of a first nonionic polymer;
0.3 wt. % to 5 wt. % of a vinylpyrrolidone dimethylamino propylacrylamide acrylate copolymer; and
1 wt. % to 10 wt. % of a cationic polymer;
1 wt. % to 20 wt. % of one or more polyols, wherein one of the one or more polyols has a greater affinity for water than at least one of the three polymers, and
0.05 wt. % to 1.2 wt. % emulsifier, wherein the emulsifier concentration is balanced stabilize a viscosity over a period of 2 to 6 weeks after mixing.

2. The composition of claim 1, wherein the polyols comprise sorbitol.

3. The composition of claim 1, wherein the first non-ionic polymer is polyvinylpyrrolidone.

4. The composition of claim 1, wherein volatile organic compounds (VOCs) comprise less than 2 wt. % of the composition.

5. The composition of claim 1, wherein the composition comprises multiple constituents that increase the viscosity of the composition over time.

6. The composition of claim 5, wherein the composition comprises multiple constituents that decrease the viscosity of the composition over time.

7. The composition of claim 6, wherein the contributions of the viscosity increasing and viscosity decreasing constituents are balanced at two weeks after mixing.

8. The composition of claim 1, further comprising a perfume.

9. The composition of claim 1, further comprising 65 to 85 wt. % water.

10. The composition of claim 1, wherein the emulsifier is a phosphate based emulsifier.

11. A method of increasing viscosity in a formulation, the method comprising:
identifying a formulation, in which the formulation includes 4 wt. % to 25 wt. % hydrophilic polymers and 1 wt. % to 12 wt. % of one or more polyols;
increasing the concentration of an emulsifier in the formulation to produce a target viscosity.

12. The method of claim 11, in which the emulsifier is a phosphate based emulsifier.

13. The method of claim 12, further comprising assessing an impact of the emulsifier on the viscosity of the formulation for a range of emulsifier concentrations.

14. The method of claim 13, in which assessing the impact of the emulsifier on the viscosity of the formulation for a range of emulsifier concentrations is performed using rotational viscometry.

15. The method of claim 14 in which the rotational viscometry is performed between 5 and 15 RPM.

16. The method of claim 11, in which the formulation further comprises a viscosity agent in the concentration of 0.02 to 2.0 wt. %.

17. The method of claim 16, in which the viscosity agent is hydroxyethylcellulose.

18. A hair styling composition, comprising:
a combination of three polymers, comprising a total of less than 20 wt. % of the composition, the polymers comprising:
4 wt. % to 8 wt. % of polyvinylpyrrolidone;
3 wt. % to 5 wt. % of a vinylpyrrolidone dimethylamino propylacrylamide acrylate copolymer; and
0.5 wt. % to 3 wt. % of a cationic polymer;
5 wt. % to 10 wt. % of one or more polyols, wherein one of the one or more polyols has a greater affinity for water than at least one of the three polymers, and
0.1 wt. % to 1.4 wt. % emulsifier, where the viscosity of the product at 6 weeks after mixing and storage at room temperature is less than 250,000 cSt as measured using rotational viscometry at 10 RPM.

19. The composition of claim 18, in which the one or more polyols comprise 6 wt. % to 8 wt. % of the composition.

20. The composition of claim 9, in which the one or more polyols comprise sorbitol.
